(19) United States
(12) Patent Application Publication
(10) Pub. No.: US 2014/0371305 A1
(43) Pub. Date: Dec. 18, 2014

(54) Mupirocin Antibiotic Composition

(71) Applicant: Daniel Banov, Sugar Land, TX (US)

(72) Inventor: Daniel Banov, Sugar Land, TX (US)

(73) Assignee: PROFESSIONAL COMPOUNDING CENTERS OF AMERICA, Houston, TX (US)

(21) Appl. No.: 13/918,376

(22) Filed: Jun. 14, 2013

(51) Int. Cl. A61K 47/10; A61K 31/351

(52) U.S. Cl. CPC A61K 47/10; A61K 31/351
USPC 514/460

(57) ABSTRACT

An antibiotic inhalation or irrigation composition for the treatment of bacterial infections especially in the respiratory tract, is provided. The antibiotic inhalation or irrigation composition may include a mixture of mupirocin as active pharmaceutical ingredient (API), and a micronized poloxamer composition as excipient. Micronized poloxamer composition may be produced by mixing poloxamer 188 and poloxamer 407 in an apparatus where a low-frequency acoustic field is applied to facilitate mixing. Antibiotic inhalation or irrigation composition may be obtained in powder or solution form. In other embodiments, antibiotic inhalation or irrigation composition may be employed to produce a topical formulation for the treatment of traumatic skin lesions. In some embodiments, other suitable poloxamers or sugar alcohols may be employed as excipients. Due to the synergistic effect of micronized poloxamer composition, antibiotic inhalation or irrigation composition may provide improved solubility and bioavailability of mupirocin, thus decreasing side effects and time of treatment.

### CROSS-REFERENCE TO RELATED APPLICATIONS

N/A

### BACKGROUND

1. Field of the Disclosure

The present disclosure relates in general to therapeutic formulations, and more particularly, to an antibiotic composition for the treatment of respiratory infections.

2. Background Information

The administration of a drug by inhalation is called a local treatment effected by a direct application of the drug to the affected area and may be expected to produce fewer side effects compared with the oral administration of a drug. However, the application of a drug by inhalation to the respiratory apparatus inclusive of naris, throat, trachea and lung, may sometimes result in insufficient absorption of the drug through the mucous membrane depending upon the drug. Therefore, inhalation treatments are at a disadvantage in being unable to achieve enough indirect remedial effect attributable to an increase of the concentration of the drug in the blood. Additionally, it is impractical to administer some drugs by inhalation, as they irritate the mucous membrane, for instance of the respiratory tracts of the bronchi, causing coughing.

For the foregoing reasons, there is a need for drugs with increased absorption through the mucous membranes of the respiratory apparatus, improved dispersibility to the peripheral airways and alveoli, and which may have reduced side effects.

### SUMMARY

The present disclosure may include a therapeutic formulation for the treatment of bacterial infections in the respiratory tract. The formulation may be employed as an antibiotic inhalation or irrigation composition. A method for preparing such composition is also described here.

The disclosed antibiotic inhalation or irrigation composition may include at least one antibiotic agent as active pharmaceutical ingredient (API) and a combination of two or more poloxamers as excipients. According to an embodiment, a suitable API may be mupirocin, while suitable poloxamers may include poloxamer 188 and poloxamer 407. Antibiotic inhalation or irrigation composition may include poloxamer 188 in concentrations of about 0.1% by weight to about 5% by weight, with about 1% by weight being preferred, poloxamer 407 in concentrations of about 0.1% by weight to about 5% by weight, with about 1% by weight being preferred.

According to an embodiment, a method for preparing antibiotic inhalation or irrigation composition is provided. The method may include combining suitable concentrations of poloxamer 188 and poloxamer 407 in an apparatus having a vessel where a low-frequency acoustic field may be applied to improve mixing of the components, and produce micronized poloxamer composition. Afterwards, micronized poloxamer composition may be combined with mupirocin. The antibiotic composition may be obtained in powder form and may be used to fill capsules, which may be later employed for inhalation or irrigation. In other embodiments, antibiotic inhalation or irrigation composition in powder form may be dissolved employing suitable solvents, such as sterile solution of sodium chloride and water, to obtain antibiotic inhalation or irrigation composition in solution form. The antibiotic inhalation or irrigation composition in solution form may be delivered to the respiratory tract using suitable devices such as aerosols, inhalers, nasal sprays, and nebulizers, among others. According to other embodiments, mupirocin may be combined with micronized poloxamer composition to produce a topical formulation for the treatment of traumatic skin lesions.

The synergistic effect of micronized poloxamer composition may provide improved solubility and bioavailability of mupirocin, thus decreasing treatment time, side effects occurrence, as well as the risk for resistance development. Antibiotic inhalation or irrigation composition may be used for treating infections caused by bacteria such as methicillin-resistant Staphylococcus aureus (MRSA), among others. In other embodiments, disclosed antibiotic composition may be employed as a topical formulation in the treatment of impetigo caused by Staphylococcus aureus and beta-hemolytic streptococci, including Streptococcus pyogenes.

Numerous other aspects, features and benefits of the present disclosure may be made apparent from the following detailed description taken together with the drawing figures.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 is a logarithmic graph illustrating the results of particle size test and depicting particle size distribution of poloxamers, according to an embodiment.

FIG. 2 is micronized poloxamer composition block diagram, in accordance with an embodiment of the present disclosure.

### DETAILED DESCRIPTION

The present disclosure is here described in detail with reference to embodiments illustrated in the drawings, which form a part here. Other embodiments may be used and/or other changes may be made without departing from the spirit or scope of the present disclosure. The illustrative embodiments described in the detailed description are not meant to be limiting of the subject matter presented here.

### Definitions

As used here, the following terms may have the following definitions:

“Active pharmaceutical ingredient (API)” refers to a substance that induces a suitable pharmacological or physiological effect, and may include agents with therapeutic, prophylactic, or cosmeceutical effects.

“Antibiotic” refers to an agent which may destroy or inhibit bacterial growth.

“Excipient” refers to a substance added to a therapeutic formulation in order to provide suitable consistency or form the formulation.

“Microprilling” refers to a process where solid spherical microprills may be produced from liquid, tablets, or encapsulated ingredients having a diameter of a few microns.

“Minimum Inhibitory Concentration (MIC)” may refer to the lowest concentration of an antimicrobial that may inhibit the visible growth of a microorganism after overnight incubation.

“Poloxamer” refers to a non-ionic triblock copolymer having surfactant properties. Poloxamers may be used as thickening agents, gel formers, co-emulsifiers, solubilizers, and consistency enhancers in creams and liquid emulsions.

### Description of the Drawings

The present disclosure may relate to a composition of ingredients that, in one embodiment may be an antibiotic inhalation or irrigation composition. The composition may include a combination of two or more poloxamers as excipients and mupirocin as an active pharmaceutical ingredient (API). According to an embodiment, disclosed composition may be employed as an inhalation or irrigation formulation for the treatment of bacterial infections in the respiratory tract. In other embodiments, antibiotic inhalation or irrigation composition may be employed to produce a topical formulation for the treatment of traumatic skin lesions.

Poloxamer Particle Size and Distribution

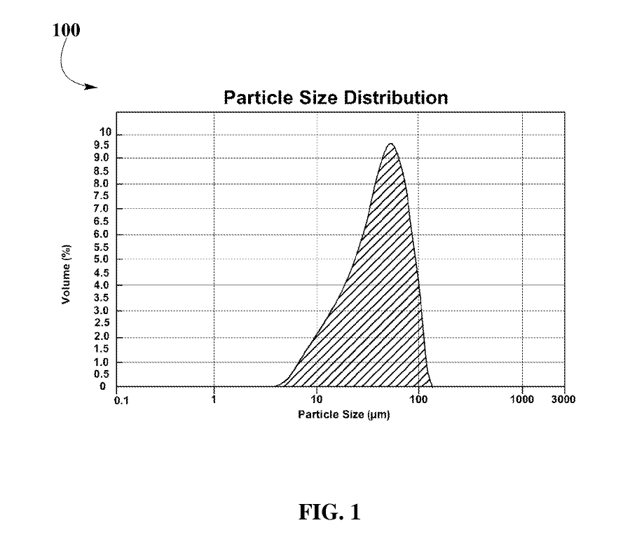

FIG. 1 is a logarithmic graph 100 illustrating the results of particle size test and depicting particle size distribution of microprilled poloxamers. Logarithmic graph 100 may show volume percentage on the “y” axis, and particle size in microns on the “x” axis. Microprilled poloxamers were used only in direct compression, where microprilled poloxamers may exhibit good blend homogeneity and segregation problem may be eliminated during direct compression. According to an embodiment, microprilled poloxamers may have an average size of about 50 μm, a small percentage of poloxamers may have a particle size between about 10 μm to about 20 μm, while the majority of poloxamers may have a particle size of about 50 μm.

Poloxamer Composition

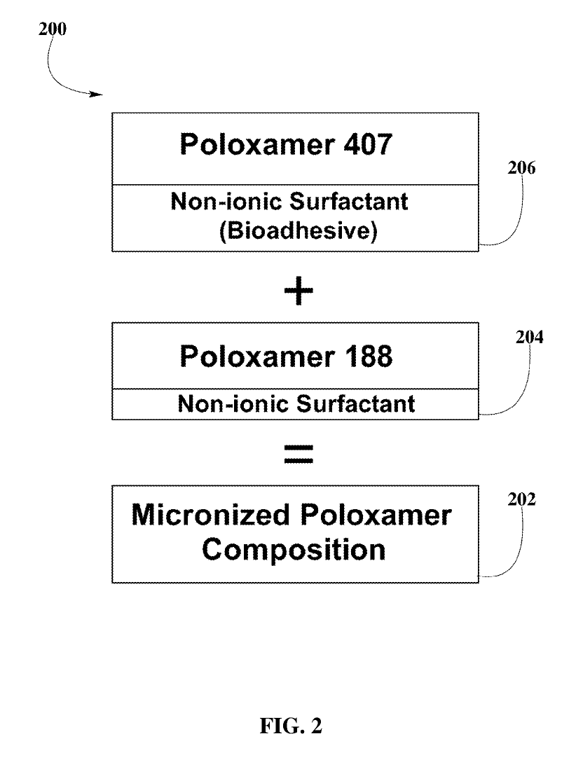

FIG. 2 is micronized poloxamer composition block diagram 200, according to an embodiment. The present disclosure may refer to an antibiotic inhalation or irrigation composition used for treating bacterial infections, especially in the respiratory tract. The antibiotic inhalation or irrigation composition may include a micronized poloxamer composition 202 as excipient. According to some embodiments, micronized poloxamer composition 202 may include poloxamer 188 204 and poloxamer 407 206. Poloxamer 188 204 may be included in concentrations of about 0.1% by weight to about 5%, with about 1% by weight being preferred, and poloxamer 407 206 in concentrations of about 0.1% by weight to about 5%, with about 1% by weight being preferred.

Micronized poloxamer composition 202 may be manufactured in an apparatus where a low-frequency acoustic field may be applied, in order to facilitate the mixing process. Suitable concentrations of poloxamer 188 204 and poloxamer 407 206 may be deposited in a vessel which may be subjected to a low-frequency acoustic field in the axial direction, resulting in second order bulk motion of the fluid, known as particle collisions. Particles in the container may be excited by collisions with the vessel base and collisions with other particles in the container that may result in harmonic vibrations of the vessel with poloxamer 188 204 and poloxamer 407 206. The particle motions may be dependent upon the vibration amplitude, frequency, and the resultant accelerations that the particles undergo. The chaotic motions created within the mixing vessel may cause a great degree of particle-to-particle disorder, microcell mixing, as well as creating bulk mixing flow in the solid-solid systems. In order to manufacture micronized poloxamer composition 202, poloxamer 188 204 and poloxamer 407 206 may be mixed with a mixing length of about 50 μm, at a mechanical resonance of about 60 Hz.

Particle size of micronized poloxamer composition 202 may range between about 30 μm to about 70 μm, where about 50 μm may be preferred. The advantages of microprilling in micronized poloxamer composition 202 may include stronger solubilization properties, controlled dissolution rate, reduction of die-wall friction, achievement of homogeneous blend, elimination of dose dumping and effectiveness as water soluble lubricant.

Furthermore, antibiotic inhalation or irrigation composition may have solubility properties dictated by the hydrophobic portion of the poloxamers. The use of poloxamers may increase the solubility of the API that is employed, thus the drug may have enhanced treatment properties. Moreover, the properties of each poloxamer may vary in terms of molecular weight, appearance, hydrophilicity/hydrophobicity, and solubility, which may be determined by the chain length of the polyxyethylene (EO-) units and polyoxypropyene (PO-) units.

Mupirocin

A suitable antibiotic agent may be employed as an API. In an embodiment, micronized poloxamer composition 202 may be combined with mupirocin to produce antibiotic inhalation or irrigation composition. In some embodiments, mupirocin may be added to micronized poloxamer composition 202 in concentrations of about 20 mg to about 100 mg, with about 80 mg being preferred.

Mupirocin is an antibiotic produced from Pseudomonas fluorescens, is bacteriostatic at low concentrations and bactericidal at high concentrations. Bacterial protein and RNA synthesis are inhibited when mupirocin reversibly binds to bacterial isoleucyl-tRNA synthetase. Bacterial isoleucyl-tRNA synthetase enzyme normally promotes the conversion of isoleucine and tRNA to isoleucyl-tRNA. The epoxide side chain of mupirocin is similar to isoleucine and competes with it for binding sites on the synthetase enzyme. The cellular concentration of isoleucyl-tRNA becomes depleted, subsequently inhibiting bacterial protein and RNA synthesis.

Mupirocin is effective against most gram-positive aerobic bacteria including Staphylococcus aureus, S. epidermidis, S. saprophyticus, most Streptococcus pneumoniae, group beta-hemolytic streptococci (S. pyogenes), group B streptococci (S. agalactiae), groups C and G streptococci, and viridans streptococci. Mupirocin is active against penicillinase-producing, nonpenicillinase-producing, and methicillin-resistant strains of Staphylococcus aureus. Additionally, mupirocin is active against some gram-positive aerobic bacilli such as Listeria monocytogenes and Erysipelothrix rhusiopathiae.

Antibiotic Inhalation or Irrigation Composition Having Mupirocin

Antibiotic inhalation or irrigation composition may be obtained in powder form. The powder may be employed to fill capsules, which may be used for inhalation or irrigation of the antibiotic composition. In other embodiments, antibiotic inhalation or irrigation composition in powder form may be dissolved in order to obtain antibiotic inhalation or irrigation composition in solution form. Suitable solvents may include sterile solution of sodium chloride, water, among others. Antibiotic inhalation or irrigation composition in solution form may include between about 2 ml to about 10 ml of solvent, and about 5 mg to about 5 g of antibiotic inhalation or irrigation composition, where about 1 g to about 2 g may be preferred. Additionally, micronized poloxamer composition 202 may optimize the particle distribution in inhalation or irrigation composition in solution form. According to an embodiment, when administered to humans, inhalation or irrigation composition may be delivered in amounts of about 2 ml to about 10 ml, where about 5 ml may be preferred.

The antibiotic inhalation or irrigation composition may be delivered to the respiratory tract employing suitable devices such as metered-dose inhalers (MDIs), dry powder inhalers, nasal sprays, aerosols, capsules, and nebulizers, among others. Employing these devices allows the patient to have a more controlled dosage of antibiotic inhalation or irrigation composition. By administering antibiotic inhalation or irrigation composition via inhalation, mupirocin may be driven directly into the lungs and less is absorbed into the bloodstream, thus increasing bioavailability of the mupirocin and decreasing treatment time.

Additionally, the antibiotic inhalation or irrigation composition may be delivered by nasal irrigation employing saline nasal sprays, and nebulizers, among others. As well, antibiotic inhalation or irrigation composition in solution form may be employed for the irrigation of traumatic skin lesions.

In other embodiments, mupirocin may be combined with micronized poloxamer composition 202 to produce a topical formulation. Mupirocin topical formulation including micronized poloxamer composition 202 may be employed in the treatment of traumatic skin lesions, such as impetigo caused by Staphylococcus aureus and beta-hemolytic streptococci, including Streptococcus pyogenes.

Furthermore, the synergistic effect of micronized poloxamer composition 202 may improve the solubility of the mupirocin, hence enhancing the action of the antibiotic inhalation or irrigation composition. Additionally, antibiotic inhalation or irrigation composition may reduce mupirocin's side effects, such as rhinitis, taste perversion, pharyngitis, burning, and cough, among others.

Moreover, micronized poloxamer composition 202 may enhance mupirocin's properties by lowering the minimum inhibitory concentration (MIC) to about 1.7 g. The MIC was tested against microorganisms such as Pseudomonas aeruginosa, and Methicillin Resistant Staphylococcus aureus, among others.

Administration of the antibiotic inhalation or irrigation composition may vary according to patient's age, weight, severity of the symptoms, and response to the treatment. In some embodiments, antibiotic inhalation or irrigation composition may be administered once or twice a day, for about 5 days.

### EXAMPLES

Example #1 is an embodiment of antibiotic inhalation or irrigation composition, where instead of employing poloxamer 188 204 and poloxamer 407 206 in micronized poloxamer composition 202, other suitable poloxamers may be used. Suitable poloxamers may include: poloxamer 101, poloxamer 105, poloxamer 108, poloxamer 122, poloxamer 124, poloxamer 181, poloxamer 182, poloxamer 183, poloxamer 184, poloxamer 185, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and combinations thereof.

Example #2 is an embodiment of micronized poloxamer composition 202, where micronized poloxamer composition 202 may be used in combination with xylitol or sugar alcohol. Xylitol may be included in amounts of about 50% by weight to about 90% by weight, most suitable being 80% by weight.

Example #3 is an application of micronized poloxamer composition 202 in combination with any suitable APIs, such as mupirocin, which may be used for treating bacterial infections in animals, applying suitable dosages according to the weight and size of the animal.

While various aspects and embodiments have been disclosed here, other aspects and embodiments may be contemplated. The various aspects and embodiments disclosed here are for purposes of illustration and are not intended to be limiting, with the true scope and spirit being indicated by the following claims.

What is claimed is:

1. A method for the prevention and treatment of infections of the respiratory tract caused by bacteria, comprising administering to a patient in need of such treatment a formulation comprising at least one active pharmaceutical ingredient and at least two poloxamers.

2. The method according to claim 1, wherein the at least one active pharmaceutical ingredient is mupirocin.

3. The method according to claim 1, wherein one of the at least two poloxamers is selected from the group consisting of poloxamer 188, poloxamer 407, and combinations thereof.

4. The method according to claim 1, wherein one of the at least two poloxamers is poloxamer 188.

5. The method according to claim 4, wherein the poloxamer 188 is about 0.1% by weight to about 5% by weight.

6. The method according to claim 4, wherein the poloxamer 188 is about 1% by weight.

7. The method according to claim 1, wherein one of the at least two poloxamers is poloxamer 407.

8. The method according to claim 7, wherein the poloxamer 407 is about 0.1% by weight to about 5% by weight.

9. The method according to claim 7, wherein the poloxamer 407 is about 1% by weight.

10. The method according to claim 1, wherein the formulation is an aerosol.

11. The method according to claim 1, wherein the formulation is an irrigant.

12. A composition for the prevention and treatment of infections of the respiratory tract caused by bacteria, comprising at least one active pharmaceutical ingredient and at least two poloxamers.

13. The composition according to claim 12, wherein the at least one active pharmaceutical ingredient is mupirocin.

14. The composition according to claim 12, wherein one of the at least two poloxamers is selected from the group consisting of poloxamer 188, poloxamer 407, and combinations thereof.

15. The composition according to claim 12, wherein one of the at least two poloxamers is poloxamer 188.

16. The composition according to claim 15, wherein the poloxamer 188 is about 0.1% by weight to about 5% by weight.

17. The composition according to claim 15, wherein the poloxamer 188 is about 1% by weight.

18. The composition according to claim 12, wherein one of the at least two poloxamers is poloxamer 407.

19. The composition according to claim 18, wherein the poloxamer 407 is about 0.1% by weight to about 5% by weight.

20. The composition according to claim 18, wherein the poloxamer 407 is about 1% by weight.
